(19) United States
(12) Patent Application Publication
(10) Pub. No.: US 2010/0298886 A1
(43) Pub. Date: Nov. 25, 2010

(54) Contact Device for Osteosynthesis

(75) Inventors: Werner Kraus, Muenchen (DE); Heribert Stephan, Muenchen (DE)

Correspondence Address: CROWELL & MORING LLP;INTELLECTUAL PROPERTY GROUP, P.O. BOX 14300, WASHINGTON, DC, 20044-4300, US

(73) Assignee: Neue Magnetodyn GmbH, Muenchen (DE)

(21) Appl. No.: 12/810,690

(22) PCT Filed: Dec. 3, 2008
(86) PCT No.: PCT/EP08/10238

§ 371 Date: Aug. 4, 2010

(30) Foreign Application Priority Data
Dec. 28, 2007 (DE) 10 2007 063 027.3

(51) Int. Cl. A61B 17/86; A61N 1/00

(52) U.S. Cl. 606/301; 607/116

(57) ABSTRACT

The invention relates to a contact device for a bone screw, wherein, in an implanted end state of the arrangement of bone screw and contact device, a first part of the contact device is arranged outside a cavity and a second part is arranged inside the cavity of the bone screw, the parts forming counterelectrodes, and the second part electrically contacts the bone screw while the first part is isolated from the bone screw, wherein the contact device has a cavity with a voltage source whose poles electrically contact each of the first part and second part directly or indirectly. In order to improve such a contact device, particularly in respect of the volume available for electrical components, it is proposed that the first part of the contact device is arranged proximally in an implanted end state of the arrangement of bone screw and contact device.

### BACKGROUND AND SUMMARY OF THE INVENTION

The invention relates to a contact device for electrically contacting an at least partially electrically conductive bone screw provided with a cavity, wherein, in an implanted final state of the arrangement of bone screw and contact device, a first part of the contact device is located outside of the cavity and a second part of the contact device is located within the cavity of the bone screw, the first part and the second part of the contact device form, at least partly, counter electrodes, and the second part of the contact device electrically contacts the bone screw while the first part of the contact device is insulated from the bone screw so that an outer surface of the bone screw acts, at least partly, as a counter electrode to the electrode formed by the first part of the contact device, the contact device being provided with a cavity and at least one voltage source being located in the cavity the poles of which electrically contact the first part and the second part of the contact device directly or indirectly, respectively.

Such contact devices are known in the field of osteosynthesis. Osteosynthesis is the load-stable fixation of the fragments of a broken or unhealthy bone in its uninjured natural form by means of implanted screws, support plates, wires, intramedullary nails, and the like. The means for osteosynthesis are generally manufactured of stainless steel or titanium alloys and enable the rapid mobilisation of the patient while simultaneously immobilising the damaged bone. Immobilisation is an indispensable prerequisite for healing.

Problematic with the rigid fixation by means of the comparably inelastic, tissue-displacing support implants is, however, the interference with the biological recovery predominantly due to the loss of blood vessels and nerves. In addition, the biomechanical quality of the support structure will suffer due to the partial physical withdrawal of its function with an increasing implantation time. When biological control decreases, however, the risk of an infection by resistant bacteria (MRSA=multi resistant staphylococcus aureus) increases. It has been shown that these bacteria can colonise the surface of metal implants in the form of an adhering biofilm and can resist the attack of antibiotics with the aid of a polysaccharide mucus layer.

These problems can be addressed by the use of electro-osteotherapy in orthopedic surgery, for example, by use of the generic contact device mentioned in the introduction. An example of such a device is described in U.S. Pat. No. 6,778,861 B1. In the magnetically induced electro-osteotherapy described therein, low-frequency electric alternating currents are induced in osteosynthesis means by exposing an affected part of the body to a magnetic alternating field. It has long been shown in numerous clinical applications of the aforementioned technology to chronic therapy-resistant, mostly infected bone defects, cysts and tumour metastases, as well as in hospital-related experimental studies that an optimum healing effect is achieved in the bone region contacting the support metal when osteosynthesis implants which act as sources of extremely low-frequency sinusoidal electric alternating potentials are used.

The transfer technique functions according to the transformer principle: the injured or unhealthy body region is permeated by a sinusoidal magnetic field having an extremely low frequency of approximately 1 to 100 Hz—preferably of 4 to 20 Hz—and a magnetic flux density of 0.5 to 5 mT (5 to 50 Gauss). This field is generated by a functional current generator in one or more—primary—outer current coils. The coils are inserted into the body part provided with the osteosynthesis means. These extremely low-frequency electromagnetic fields penetrate both tissue, including possible clothing and casts, as well as the non-magnetic (austenitic) support metals of the osteosynthesis with little to no loss. A—secondary—coil arrangement, the so-called transducer, is implanted in electric contact with the support metals. The electric potentials induced in the transducer are thus brought to bear in the area of the bone lesion as well as generally in the tissue adjacent to the osteosynthesis means.

With this technique of inductively transmitting therapeutically effective electric potentials to the constituent parts of the osteosynthesis, the infection risk is avoided by percutaneous current lines, and the treatment parameters, namely electric voltage, frequency, intensity, signal waveform and the treatment time, can be determined by means of the indication-specific programming of a functional current generator of the induced magnetic field.

Aside from the electro-osteosynthesis based upon magnetic induction, it was already suggested to arrange the energy source in the implant so that no complex external apparatuses are required to generate the recuperative electric fields and, further, so that the electric fields are, in the ideal case, permanently present, i.e. not only present when the patient is inside of the external magnetic field. It is, however, problematic with such solutions that an increased volume for the energy source has to be provided in the implant which is naturally not readily available due to the medical constraints. If, for example, FIG. 4 of U.S. Pat. No. 6,778,861 B1 is regarded, the volume for the energy source is limited by at least the length and the diameter of the insertion inserted into the bone screw.

The object of the present invention is to further develop a generic contact device so that an increased volume is available for the accommodation of electric components. Further, the manageability and the flexible usage of the contact device during the surgery are to be improved, as well as its the biologic effect, the therapeutic effectiveness, and the profitability.

The invention is based on the generic contact device in that the first part of the contact device is arranged proximally in an implanted final state of the arrangement of bone screw and contact device. With this arrangement of the first part of the contact device located outside of the bone screw, an increased volume is available within the contact device. The contact device may be inserted into an already-implanted bone screw in a simple manner during surgery without requiring particular preparatory measures. In particular, the tip existing on the distal end of the arrangement present in FIG. 4 of the U.S. Pat. No. 6,778,861 B1 is omitted, and no pre-drilling or other measures for ensuring sufficient electrical contact of the no longer existent tip of the contact device have to be taken. Practically any conventional cannulated screw can be provided with the contact device. The surgeon can decide whether he wishes to implant the contact device or not after the bone screw has already been inserted. This is particularly relevant if a plurality of bone screws is used to fix a bone plate which will become a part of the electrified components. One or more bone screws may then be provided with the contact device according to the invention while the other bone screws may remain without a contact device. In any case, the decision regarding which screws are to be provided with the contact device can be made after the arrangement of bone plate and bone screws is fully implanted. With the arrangement according to the invention, the entire bone screw preferably becomes a cathode, while the first part of the contact device located outside of the bone screw forms the anode, at least in part. In this manner, the magnetically induced osteogenesis is concentrated to the stabilising area of the bone screw, i.e. the screw shaft, as the osteogenesis depends on the polarity of the respective electrodes, i.e. it is promoted at the cathode and impeded at the anode. Therefore, bone formation is impeded or avoided in the area of the proximal part of the contact device, and/or osteolysis is brought about while in the area of the fracture, bone formation is boosted in a desired manner. Further, the explanation of the bone screw is simplified as the contact device can be simply be detached without it being impeded by bone tissue.

It is intended that the contact device can be inserted into the bone screw. Based upon this advantage, the contact device according to the invention can be inserted into practically any bone screw since no particular means for receiving the contact device has to be provided at the inner wall of the bone screw.

In this regard, it is particularly advantageous that the second part of the contact device contacts the bone screw via at least one spring contact. One or more spring contacts are in contact with the contact device during its insertion and then support themselves after a specific insertion position. They continue to support themselves in the final state of the arrangement of bone screw and contact device, at the inner wall of the hollow bone screw. The contact device may from the outset be provided with one or more of said spring contacts. It is, however, also feasible that the spring contact(s) act, more or less, as adapters between the contact device and the bone screw so that contact devices identical in construction can act together with different bone screws owing to the interposition of different spring contacts.

According to another embodiment of the invention, it is intended that the contact device can be screwed into the bone screw. In this case, the contact device has to be provided with a male thread, and a female thread must be provided in the cavity of the bone screw.

It is particularly advantageous that the at least one voltage source comprises an accumulator. Sufficient volume is now available for such an accumulator, for example a lithium ion accumulator, particularly inside of the first part of the contact device located outside of the bone screw.

It may also be provided that the at least one voltage source comprises a capacitor. For example, a so-called supercap may be provided for storing energy.

It is further advantageous that the at least one voltage source comprises a coil.

It is then particularly advantageous that the coil is connected in parallel with the accumulator and/or the capacitor. Even though an accumulator or a capacitor used as an energy source can do without a coil, and inversely, a coil for electrifying the bone screw as such is sufficient for absorbing the energy of an external magnetic field, a combination of coil and accumulator and/or capacitor may be particularly useful. Namely, the coil can then be used for charging the energy storage(s) by way of magnetic induction so that the energy required for the electrification of the device is, in fact, provided by an external magnetic field but is also available in the absence of an external magnetic field. Therefore, it can, for example, suffice that an implant is exposed once a day for a short period of time to an external alternating magnetic field by which the energy storage(s) is/are charged and that the stored energy is then available for the remainder of the day.

It may additionally be advantageous that a function generator is located in the cavity of the contact device. The generator preferably generates sinusoidal or similar alternating voltages. If the energy is provided by an accumulator inside of the contact device, a direct voltage is available which is capable of electrifying a bone screw in a suitable manner. However, it may be desired to provide an alternating electric voltage in this way in order to generate electric conditions comparable to those known from the known alternating field therapy, namely with an external magnetic field and an internal repeating coil. In connection with such alternating voltages, however, it should be kept in mind that they generate an at least predominantly positive polarity at the outer part of the contact device, while the screw shaft is on an at least predominantly negative potential. In this way, bone growth is promoted in the damaged area of the bone, and unnecessary ingrowth of the outer part of the contact device is impeded or avoided. The provision of said potentials may be implemented by connecting a diode and potentially a smoothing capacitor in parallel with the voltage source formed by the function generator.

It may be particularly advantageous that a test circuit and a circuitry for high frequency identification for detecting and transmitting signals are located in the cavity of the contact device, and the signals, in particular, correspond to physiologically relevant parameters. With the measuring device, for example, an impedance depending on the condition of the tissue or a pH-value can be measured. Likewise, the applied voltage can be continuously monitored. The corresponding signals may then be transmitted to an external analysing device via the circuitry for high frequency identification (RFID).

It may be advantageous that the contact device engages with the bone screw by means of a latch or snap-in connection for adopting a final implanted state of the arrangement of bone screw and contact device. In this way, the surgeon has a further indication for the correct arrangement of the contact device inside of the bone screw even under difficult conditions. The latch or snap-in connection may also prevent an unintended later displacement of the contact device with respect to the bone screw.

The invention further relates to an osteosynthesis device comprising a bone screw according to the invention.

The invention will now be explained by way of example on the basis of particularly preferred embodiments with reference to the accompanying drawings:

### BRIEF DESCRIPTION OF THE DRAWINGS

FIG. 1 is a cross section led in the axial direction through a first embodiment of an arrangement of bone screw and contact device, and

FIG. 2 is a cross section led in the axial direction through a second embodiment of an arrangement of bone screw and contact device.

### DETAILED DESCRIPTION OF THE DRAWINGS

In the following description of the preferred embodiments of the present invention the same numerals designate identical or comparable components.

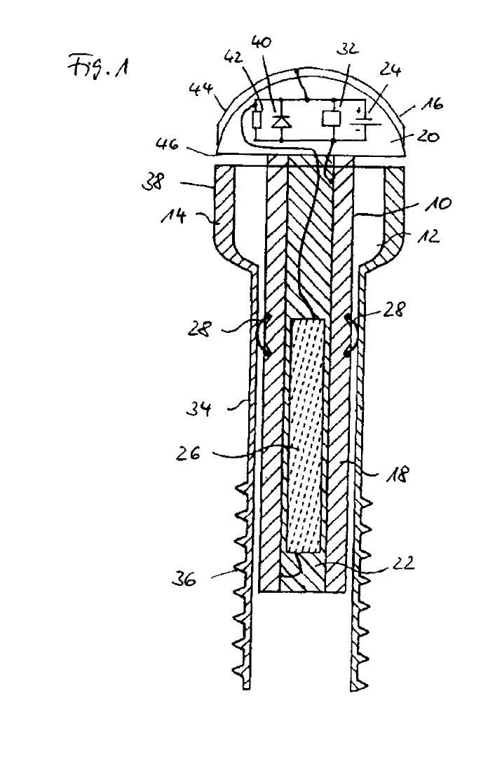

FIG. 1 shows a cross section in the axial direction through a first embodiment of an arrangement of bone screw and contact device. A bone screw 14 and a contact device 10 inserted in a cavity 12 of the bone screw 14 are shown.

The bone screw 14 comprises a screw shaft 34 and a threaded section 36 provided at the screw shaft 34. At the proximal end of the screw shaft 34, a screw head 38 is attached which is preferably provided with a device in which a tool for screwing in the bone screw 14 may engage. For example, the cavity 12 of the bone screw 14 is limited by an internal hexagon or comparable positive fit and/or force closure devices in the area of the screw head 38.

The contact device 10 comprises a proximally arranged cap-shaped first part 16 and a distally arranged elongated second part 18. The distal second part 18 protrudes into the cavity 12 of the bone screw 14. Electrically conductive spring contacts 28 are provided for mechanically and electrically contacting the contact device 10 and the bone screw 14. A plurality of spring contacts may also be provided at various axial coordinates other than shown here in order to enhance the mechanic and electric contact. The cap-shaped first part 16 of the contact device 10 contains a cavity 20 in which the various electric components are located, in the present embodiment an accumulator 24, a circuitry 32 for high frequency identification 32 (RFID), a diode 40, and an ohmic resistance 42. The accumulator 24 provides electric power, and the cathode thereof is electrically connected to the elongated second part 18 of the contact device 10, and the anode thereof contacts an electrically conductive outer shell 44 of the cap-shaped first part 16 of the contact device 10. By electrically contacting the elongated second part 18 of the contact device 10 and the bone screw 14, the latter will become the cathode while the outer electrically conductive shell 44 of the cap-shaped first part 16 the contact device 10 forms the anode.

A short circuit between said two counter electrodes is to be prevented, and to this end it may be intended that the cap-shaped first part 16 of the contact device 10 comprises an electrically insulating region 46 forming the distal boundary of the cap-shaped first part 16 the contact device 10. The insulating region 46 may, for example, be realised by a DLC layer (DLC=diamond-like carbon). Alternatively or in addition, further insulating means may be provided between the cap-shaped first part 16 of the contact device 10 and the bone screw 14. Said insulating means can be formed together with the bone screw 14, together with the cap-shaped first part 16 of the contact device 10 or even as a separate component.

The final state of the implanted arrangement of contact device 10 and bone screw 14 can be definitely defined, for example, by a stopper which can, for example, be formed by a contact between the insulating part 46 of the cap-shaped first part 16 of the contact device, or which may also be realised by a latch or snap-in connection. The final state of the implanted arrangement of contact device 10 and bone screw 14 may also be variable so that the surgeon can decide during the operation how far to insert the contact device 10 into the bone screw.

It can be particularly advantageous to embed the electric components in the cavity 20 of the cap-shaped first part 16 of the contact device 10 in an electrically insulating mass, for example a tissue-compatible cast resin. The same applies to the cavity 22 located within the elongated second part 18 of the contact device 10. In the present embodiment, a coil 26 preferably having a magnetically soft core is located within said cavity 22. Said coil serves as an energy provider for charging the accumulator 24, when the arrangement of bone screw 14 and contact device 10 is located in an outer alternating magnetic field. To this end, the coil 26 is electrically connected in parallel to the accumulator 24; in other words, a pole of the coil is connected to the anode of the accumulator 24 while the other pole of the coil is connected to the cathode of the accumulator 24, which is here under interposition of the electrically conductive elongated second part 18 of the contact device 10. A direct contact of the electric components is also possible.

The other electric components located in the first part 16 of the contact device 10 serve different purposes. The arrangement of the RFID 32 in the cap-shaped first part 16 of the contact device 10 is to be understood schematically. It is important that said RFID 32 can transmit signals based on output signals from internal measuring circuits to an external analysing device. The diode 40 serves the rectification of the voltage, namely when it is provided directly through the coil 26. The ohmic resistance 42 is a substitute for various other electric components through which the voltage characteristics can be modified.

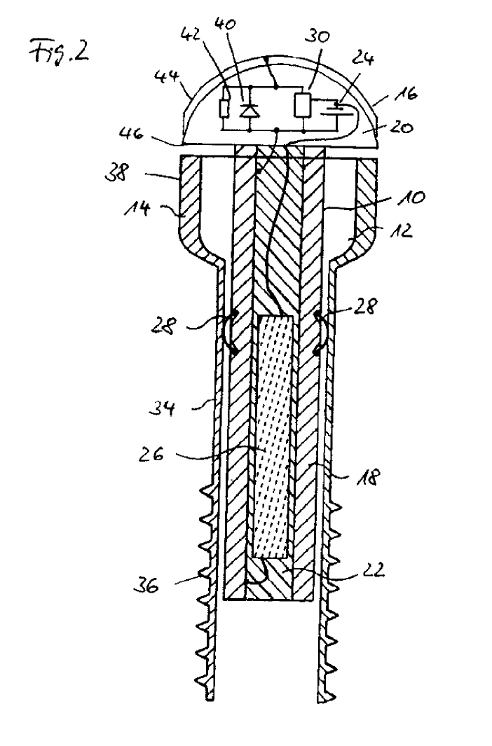

FIG. 2 shows a cross section led in the axial direction through a second embodiment of an arrangement of a bone screw and a contact device. Here a function generator 30 is employed which is supplied with power by the accumulator 24 via its input terminals while its output terminals contact the two parts 16, 18 of the contact device 10. The diode, on the other hand, can make sure that the proximal cap-shaped first part 16 has a predominantly positive potential while the bone screw 14 receives a predominantly cathodic component.

As explained in connection with FIG. 1, an RFID serving the telemetry may also be provided in the embodiment shown here and comprise a function generator 30.

The features of the invention disclosed in the above description, in the drawings as well as in the claims may be important for the realisation of the invention individually or in any combination.

### LIST OF REFERENCE NUMERALS

Contact device
12 Cavity
14 Bone screw
16 First part
18 Second part
20 Cavity
22 Cavity
24 Accumulator
26 Coil
28 Spring contact
30 Function generator
32 Circuitry
34 Screw shaft
36 Thread
38 Screw head
40 Diode
42 Ohmic resistance
44 Conductive hull
46 Insulating housing

1. Contact device for electrically contacting an at least partly electrically conductive bone screw provided with a cavity, wherein in an implanted final state of the arrangement of bone screw and contact device a first part of the contact device is arranged outside of the cavity and a second part (18) of the contact device is located inside of the cavity of the bone screw, the first part and the second part of the contact device, at least partly, forming counter electrodes, and the second part of the contact device electrically contacting the bone screw while the first part of the contact device is insulated with respect to the bone screw so that an outer surface of the bone screw acts, at least partly, as a counter electrode to the electrode formed by the first part of the contact device, wherein the contact device comprises a cavity and at least one voltage source the poles of which directly or indirectly electrically contact the first part and the second part of the contact device, respectively, is located in the cavity, and wherein the first part of the contact device is arranged proximally in an implanted final state of the arrangement of bone screw and contact device.

2. Contact device according to claim 1, wherein the contact device can be inserted into the bone screw.

3. Contact device according to claim 1, wherein the second part of the contact device contacts the bone screw via at least one spring contact.

4. Contact device according to claim 1, wherein the contact device can be screwed into the bone screw.

5. Contact device according to claim 1, wherein the at least one voltage source comprises an accumulator.

6. Contact device according to claim 1, wherein the at least one voltage source comprises a capacitor.

7. Contact device according to claim 1, wherein the at least one voltage source comprises a coil.

8. Contact device according to claim 7, wherein the coil is connected in parallel to at least one of the accumulator and the capacitor.

9. Contact device according to claim 1, wherein a function generator is located in the cavity of the contact device.

10. Contact device according to claim 1, wherein a test circuit and a circuitry for high frequency identification for detecting and transmitting signals are located in the cavity of the contact device, and the signals correspond to physiologically relevant parameters.

11. Contact device according to claim 1, wherein the contact device takes a latch or snap-in connection with the bone screw for adopting an implanted final state of the arrangement of bone screw and contact device.

12. Osteosynthesis device comprising a bone screw and a contact device according to claim 1.
